(19) United States
(12) Patent Application Publication
(10) Pub. No.: US 2018/0369606 A1
(43) Pub. Date: Dec. 27, 2018

(54) SYSTEMS AND METHODS FOR MAKING AND USING IMPLANTABLE OPTICAL STIMULATION LEADS AND ASSEMBLIES

(71) Applicant: Boston Scientific Neuromodulation Corporationd, Valencia, CA (US)

(72) Inventors: Tianhe Zhang, Studio City, CA (US); Rosana Esteller, Santa Clarita, CA (US)

(21) Appl. No.: 16/017,500

(22) Filed: Jun. 25, 2018

Related U.S. Application Data
(60) Provisional application No. 62/524,910, filed on Jun. 26, 2017.

(51) Int. Cl. A61N 5/06; A61B 5/00; A61B 5/1482

(52) U.S. Cl. CPC A61N 5/0622; A61B 5/6868; A61B 5/4836; A61N 2005/067; A61N 2005/0653; A61N 2005/063; A61B 5/1482

(57) ABSTRACT

An optical stimulation system includes a control module coupleable to a lead having a light emitter and a sensing electrode. The light emitter emits light having one or more wavelengths that activate light-sensitive neurons within a target stimulation location. The light-sensitive neurons generate either an excitatory response or an inhibitory response when activated depending on the wavelength of the emitted light. The sensing electrode senses electrical activity from the activated light-sensitive neurons concurrently with emission of the light from the light emitter. The control module directs emission of light from the light emitter using stimulation parameters. The control module includes a closed-loop feedback subsystem for adjusting at least one of the stimulation parameters based, at least in part, on electrical activity of the activated light-sensitive neurons sensed by the sensing electrode.

### CROSS-REFERENCE TO RELATED APPLICATIONS

This application claims the benefit under 35 U.S.C. § 119(e) of U.S. Provisional Patent Application Ser. No. 62/524,910, filed Jun. 26, 2017, which is incorporated herein by reference.

### FIELD

The present invention is directed to the area of implantable optical stimulation systems and methods of making and using the systems. The present invention is also directed to implantable optical stimulation leads having closed-loop feedback subsystems for controlling optical stimulation, as well as methods of making and using the leads and optical stimulation systems.

### BACKGROUND

Implantable optical stimulation systems can provide therapeutic benefits in a variety of diseases and disorders. For example, optical stimulation can be applied to the brain either externally or using an implanted stimulation lead to provide, for example, deep brain stimulation, to treat a variety of diseases or disorders. Optical stimulation may also be combined with electrical stimulation.

Stimulators have been developed to provide therapy for a variety of treatments. A stimulator can include a control module (for generating light or electrical signals sent to light sources in a lead), one or more leads, and one or more light sources coupled to, or disposed within, each lead. The lead is positioned near the nerves, muscles, or other tissue to be stimulated.

### BRIEF SUMMARY

One embodiment is an optical stimulation system including an optical stimulation lead and a control module coupleable to the optical stimulation lead. The optical stimulation lead has a lead body, a light emitter, and a sensing electrode. The lead body has a distal portion and a proximal portion. The light emitter is disposed along the distal portion of the lead body and is configured and arranged to emit light having one or more wavelengths that activate light-sensitive neurons. The light-sensitive neurons generate either an excitatory response or an inhibitory response when activated depending on the wavelength of the emitted light. The sensing electrode is disposed along the distal portion of the lead body and is configured and arranged to sense electrical activity from the activated light-sensitive neurons concurrently with emission of the light from the light emitter. The control module is configured and arranged to direct the emission of the light from the light emitter using a set of stimulation parameters. The control module includes a closed-loop feedback subsystem configured and arranged for adjusting at least one stimulation parameter of the set of stimulation parameters based, at least in part, on electrical activity of the activated light-sensitive neurons sensed by the sensing electrode. In at least some embodiments, the light emitter is configured and arranged to emit light having one or more wavelengths that activate light-sensitive neurons within a target stimulation location into which genetic agents were previously introduced.

In at least some embodiments, the sensing electrode is configured and arranged to sense changes in electrical activity from the activated light-sensitive neurons in response to the emitted light. In at least some embodiments, the sensing electrode is configured and arranged to sense at least one of a level of neuronal activation or a neuronal firing rate of the light-sensitive neurons in response to the emitted light. In at least some embodiments, the sensing electrode is configured and arranged to sense at least one surrogate electrical signal from the light-sensitive neurons in response to the emitted light, the surrogate electrical signal usable for determining at least one of a level of neuronal activation or a neuronal firing rate of the light-sensitive neurons in response to the emitted light. In at least some embodiments, the at least one surrogate electrical signal comprises one of an evoked a compound action potential, local field potential, a multiunit activity signal, an electroencephalogram signal, an electrophysiology signal, an electrospinogram signal, or an electroneurogram signal.

In at least some embodiments, the set of stimulation parameters includes at least one of intensity, pulse width, pulse frequency, cycling, or electrode stimulation configuration. In at least some embodiments, the optical stimulation lead is one of a percutaneous lead or a paddle lead. In at least some embodiments, the optical stimulation system further includes a programmer coupled to the control module, the programmer configured and arranged for implementing at least one stimulation parameter of the set of stimulation parameters.

In at least some embodiments, the closed-loop feedback subsystem includes one of a proportional controller, a proportional integral controller, a proportional derivative controller, or a proportional-integral-derivative controller. In at least some embodiments, the closed-loop feedback subsystem includes a smart machine learning module.

In at least some embodiments, the light emitter is side-facing with respect to the lead body. In at least some embodiments, the light emitter is forward-facing with respect to the lead body.

In at least some embodiments, the optical stimulation system further includes a light source in communication with the control module, the light source configured and arranged to generate light emitted by the light emitter. In at least some embodiments, the light source is disposed in the control module. In at least some embodiments, the light source is disposed in the lead.

Another embodiment is a method for optically stimulating a patient. The method includes advancing the optical stimulation lead of the optical stimulation system described above in proximity to a first target stimulation location within the patient, the first target stimulation location containing light-sensitive neurons, the light-sensitive neurons generating either an excitatory response when activated by light of a first wavelength, or an excitatory response when activated by light of a second wavelength; emitting light at either the first wavelength or the second wavelength from the light emitter of the optical stimulation lead towards the first target stimulation location; sensing, using the sensing electrode of the optical stimulation lead, electrical activity from the light-sensitive neurons at the first target stimulation location; and adjusting, using the closed-loop feedback subsystem of the optical stimulation system, at least one stimulation parameter of the set of stimulation parameters of the emitted light in response to changes in electrical activity sensed by the sensing electrode. In at least some embodiments, advancing the optical stimulation lead of the optical stimulation system described above in proximity to a first target stimulation location within the patient, the first target stimulation location containing light-sensitive neurons includes advancing the optical stimulation lead in proximity to a first target stimulation location containing light-sensitive neurons into which genetic agents were previously introduced.

In at least some embodiments, adjusting, using the closed-loop feedback subsystem of the optical stimulation system, at least one stimulation parameter of the set of stimulation parameters of the emitted light in response to changes in electrical activity sensed by the sensing electrode includes adjusting intensity of the emitted light.

In at least some embodiments, sensing, using the sensing electrode of the optical stimulation lead, electrical activity from the light-sensitive neurons at the first target stimulation location includes sensing at least one of a level of neuronal activation or neuronal firing rate of the light-sensitive neurons within the first target stimulation location. In at least some embodiments, sensing, using the sensing electrode of the optical stimulation lead, electrical activity from the light-sensitive neurons at the first target stimulation location includes sensing at least one of an evoked compound action potential, a local field potential, a multiunit activity signal, an electroencephalogram signal, an electrophysiology signal, an electrospinogram signal, or an electroneurogram signal within the first target stimulation location. In at least some embodiments, sensing, using the sensing electrode of the optical stimulation lead, electrical activity from the light-sensitive neurons at the first target stimulation location includes sensing electrical activity from the light-sensitive neurons at the first target stimulation location concurrently with emission of light from the light emitter.

In at least some embodiments, advancing the optical stimulation lead of the optical stimulation system described above in proximity to a first target stimulation location within the patient includes advancing the optical stimulation lead in proximity to each of a first target stimulation location and a second target stimulation location within the patient, the first target stimulation location and the second target stimulation location each containing light-sensitive neurons, the light-sensitive neurons generating either an excitatory response when activated by light of a first wavelength, or an inhibitory response when activated by light of a second wavelength; and emitting, concurrently, light at either the first wavelength or the second wavelength from the light emitter of the optical stimulation lead towards the first target stimulation location and the second target stimulation location.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of an optical stimulation system that includes a lead coupled to a control module, according to the invention;

FIG. 2A is a schematic side view of one embodiment of the control module of FIG. 1 configured and arranged to couple to an elongated device, according to the invention;

FIG. 2B is a schematic side view of one embodiment of a lead extension configured and arranged to couple the elongated device of FIG. 2A to the control module of FIG. 1, according to the invention;

FIG. 3 is a schematic overview of one embodiment of components of a stimulation system, including an electronic subassembly disposed within a control module, according to the invention;

FIG. 4 is a schematic overview of one embodiment of an optical stimulation lead with a light source, a processor, and a closed-loop feedback subsystem, according to the invention;

FIG. 5 is a schematic side view of one embodiment of a distal portion of an optical stimulation lead and an activation field generated by light emitters of the optical stimulation lead, according to the invention;

FIG. 6A is a schematic top view of one embodiment of a light emitter and a sensing electrode disposed in a paddle body and aligned beneath an optically-transparent region formed in the paddle body, according to the invention;

FIG. 6B is a schematic top view of one embodiment of the paddle body of FIG. 6A, with sensing electrodes disposed on or in a paddle body, according to the invention;

FIG. 7A is a schematic side view of one embodiment of a distal portion of a lead with segmented optically-transparent regions formed in a body of the lead, according to the invention;

FIG. 7B is a schematic side view of one embodiment of a distal portion of the lead of FIG. 7A with segmented optically-transparent regions and a distal-tip optically-transparent region formed in a body of the lead, according to the invention;

FIG. 8A is a schematic side view of one embodiment of a distal portion of a lead with ring-shaped optically-transparent regions formed in a body of the lead, according to the invention;

FIG. 8B is a schematic side view of one embodiment of a distal portion of the lead of FIG. 8A with ring-shaped optically-transparent regions and a distal-tip optically-transparent region formed in a body of the lead, according to the invention;

FIG. 9A is a schematic side view of one embodiment of a distal portion of a lead with segmented optically-transparent regions and ring-shaped optically-transparent regions formed in a body of the lead, according to the invention; and

FIG. 9B is a schematic side view of one embodiment of a distal portion of the lead of FIG. 9A with segmented optically-transparent regions, ring-shaped optically-transparent regions, and a distal-tip optically-transparent region formed in a body of the lead, according to the invention.

### DETAILED DESCRIPTION

The present invention is directed to the area of implantable optical stimulation systems and methods of making and using the systems. The present invention is also directed to implantable optical stimulation leads having closed-loop feedback subsystems for controlling optical stimulation, as well as methods of making and using the leads and optical stimulation systems.

In some embodiments, the implantable optical stimulation system only provides optical stimulation. Examples of optical stimulation systems with leads are found in, for example, U.S. patent application Ser. No. 15/450,969 which is incorporated by reference in its entirety. In other embodiments, the stimulation system can include both optical and electrical stimulation. In at least some of these embodiments, the optical stimulation system can be a modification of an electrical stimulation system to also provide optical stimulation. Suitable implantable electrical stimulation systems that can be modified to also provide optical stimulation include, but are not limited to, a least one lead with one or more electrodes disposed along a distal portion of the lead and one or more terminals disposed along the one or more proximal portions of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; and 2013/0105071; and U.S. patent application Ser. Nos. 12/177,823 and 13/750,725, all of which are incorporated by reference in their entireties.

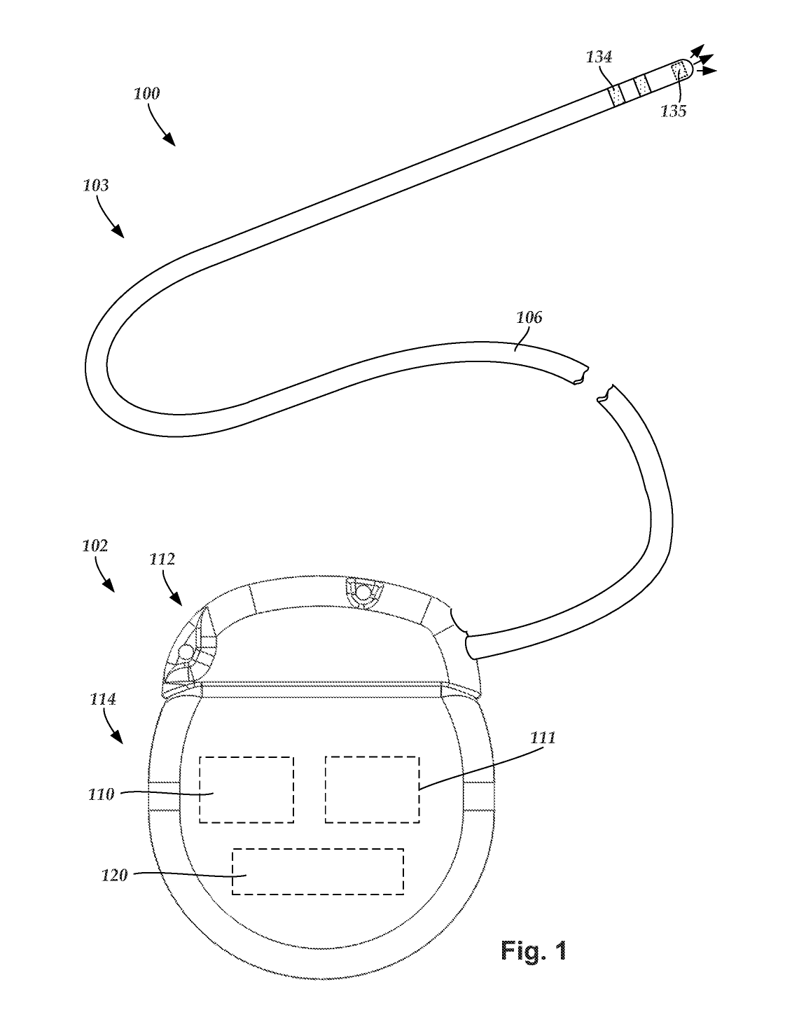

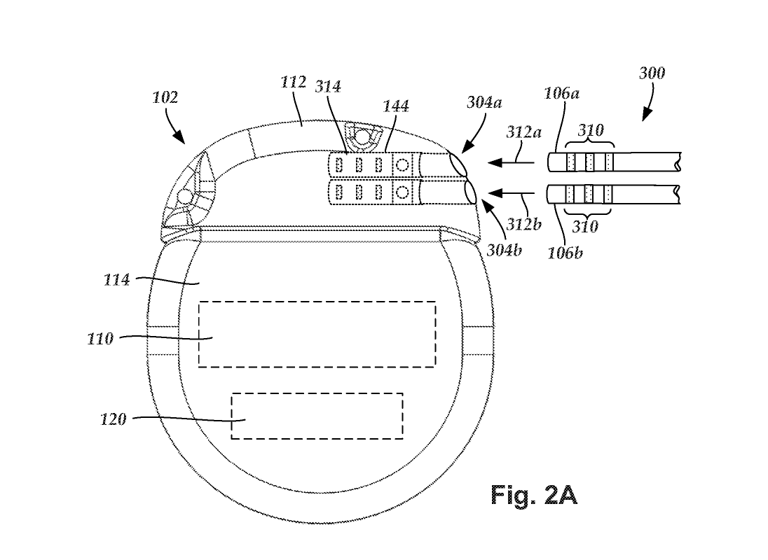

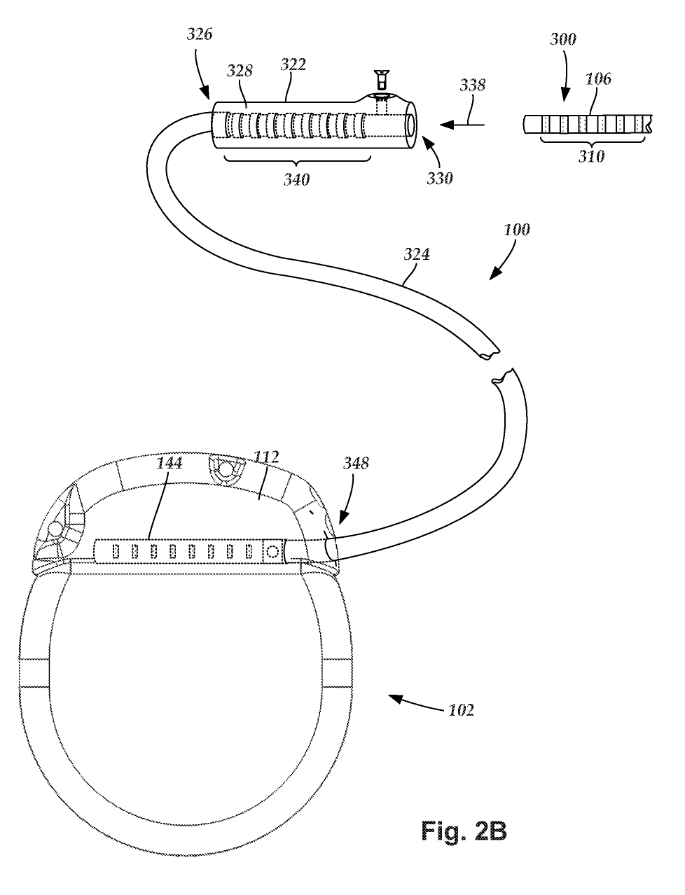

FIG. 1 illustrates schematically one embodiment of an optical stimulation system 100. The optical stimulation system includes a control module (e.g., a stimulator) 102 and a lead 103 coupleable to the control module 102. The lead 103 includes one or more lead bodies 106. In FIG. 1, the lead 103 is shown having a single lead body 106. In FIG. 2B, the lead 103 includes two lead bodies. It will be understood that the lead 103 can include any suitable number of lead bodies including, for example, one, two, three, four, five, six, seven, eight or more lead bodies 106.

At least one light emitter 135 is provided along a distal portion of the lead 103. The light emitter 135 can be a light source, such as a light-emitting diode (“LED”), laser diode, organic light-emitting diode (“OLED”), or the like, or can be a terminus of a light transmission element, such as an optical fiber, in which case the light source is distant from the distal portion of the lead (for example, in the control module or in a proximal portion of the lead). The lead also includes electrodes 134 disposed along the lead body 106, and one or more terminals (e.g., 310 in FIG. 2A-2B) disposed along each of the one or more lead bodies 106 and coupled to the electrodes 134 by conductors (not shown). In at least some embodiments, one or more terminals (e.g., 310 in FIG. 2A-2B) may also be used to convey electrical signals to a light source that acts as the light emitter 135 by conductors (not shown) extending along the lead.

The electrodes 134 include at least one sensing electrode for sensing electrical activity. Optionally, the one or more electrodes 134 can include at least one stimulation electrode for providing electrical stimulation in addition to, or in lieu of, optical stimulation provided via the at least one light emitter 135.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. In at least some embodiments, at least one of the electrodes 134 is formed from an optically-transparent material. Any suitable number of electrodes 134 can be disposed on the lead including, for example, one, two, three, four, five, six, seven, eight, nine, ten, eleven, twelve, fourteen, sixteen, twenty-four, thirty-two, or more electrodes 134.

The lead 103 can be coupled to the control module 102 in any suitable manner. In some embodiments, the lead is permanently attached to the control module 102. In other embodiments, the lead can be coupled to the control module 102 by a connector (e.g., connector 144 of FIG. 2A). In FIG. 2A, the lead 103 is shown coupling directly to the control module 102 through the connector 144. In at least some other embodiments, the lead 103 couples to the control module 102 via one or more intermediate devices, as illustrated in FIG. 2B. For example, in at least some embodiments one or more lead extensions 324 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the stimulation system 100 includes multiple elongated devices disposed between the lead 103 and the control module 102, the intermediate devices may be configured into any suitable arrangement.

The control module 102 can include, for example, a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

In some embodiments, the control module 102 also includes one or more light sources 111 disposed within the sealed electronics housing 114. In alternate embodiments, the one or more light sources 111 are external to the control module. The one or more light sources can be, for example, a light-emitting diode (“LED”), laser diode, organic light-emitting diode (“OLED”), or the like. When the control module 102 includes multiple light sources, the light sources can provide light in at a same wavelength or wavelength band or some, or all, of the light sources can provide light at different wavelength or different wavelength bands. When the one or more light sources 111 are external to the lead(s), the light emitted by the light sources can be directed to one or more optical fibers (for example, optical fibers 420a, 420b in FIG. 4) or other light-transmitting body. The optical fiber, or a series of optical fibers, can transmit the light from the one or more light sources 111 through the control module 102 and lead 103 to the light emitter 135 (which can be terminus of the optical fiber). In at least some embodiments, the optical fiber is a single mode optical fiber. In other embodiments, the optical fiber is a multi-mode optical fiber. In some embodiments, the system includes a single optical fiber. In other embodiments, the system may employ multiple optical fibers in series or in parallel.

In other embodiments, the light emitter 135 can also be the light source (a light-emitting diode (“LED”), laser diode, organic light-emitting diode (“OLED”), or the like), or a combination of light sources, with conductors extending along the lead 103 and coupled to the electronic subassembly 110 to provide signals and power for operating the light source. In yet other embodiments, the light source can be disposed elsewhere in the control module 102, on the lead 103, in another element such as a lead extension, splitter, adaptor, or other stand-alone element.

The stimulation system or components of the stimulation system, including the lead 103 and the control module 102, are typically implanted into the body of a patient. The stimulation system can be used for a variety of applications including, but not limited to brain stimulation, deep brain stimulation, neural stimulation, spinal cord stimulation, muscle stimulation, sacral nerve stimulation, dorsal root ganglion stimulation, peripheral nerve stimulation, and the like.

The one or more lead bodies 106 are made of a non-conductive, biocompatible material such as, for example, silicone, polyurethane, polyether ether ketone (“PEEK”), epoxy, and the like or combinations thereof. The one or more lead bodies 106 may be formed in the desired shape by any process including, for example, molding (including injection molding), casting, and the like.

One or more terminals (e.g., 310 in FIGS. 2A-2B) are typically disposed along the proximal end of the one or more lead bodies 106 of the stimulation system 100 (as well as any splitters, lead extensions, adaptors, or the like) for electrical connection to corresponding connector contacts (e.g., 314 in FIGS. 2A-2B). The connector contacts are disposed in connectors (e.g., 144 in FIGS. 1-2B; and 322 FIG. 2B) which, in turn, are disposed on, for example, the control module 102 (or a lead extension, a splitter, an adaptor, or the like). Electrically conductive wires, cables, or the like (not shown) extend from the terminals to the light emitter 135 or electrodes 134.

The electrically-conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 2A is a schematic side view of one embodiment of a proximal portion of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 2B, an adaptor, or the like or combinations thereof), or a combination thereof.

The control module connector 144 defines at least one port into which a proximal end of the elongated device 300 can be inserted, as shown by directional arrows 312a and 312b. In FIG. 2A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 300 is inserted into the ports 304a and 304b, the connector contacts 314 can be aligned with a plurality of terminals 310 disposed along the proximal end(s) of the elongated device(s) 300 to electrically couple the control module 102 to the electrodes (134 of FIG. 1) disposed on the paddle body 104 of the lead 103. Each of the terminals 310 can couple to the light emitter 135 or one or more of the electrodes 134. Examples of connectors in control modules are found in, for example, U.S. Pat. Nos. 7,244,150 and 8,224,450, which are incorporated by reference.

FIG. 2B is a schematic side view of another embodiment of the stimulation system 100. The stimulation system 100 includes a lead extension 324 that is configured and arranged to couple one or more elongated devices 300 (e.g., one of the lead bodies 106 of FIG. 1, a splitter, an adaptor, another lead extension, or the like or combinations thereof) to the control module 102. In FIG. 2B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144 (e.g., the ports 304a and 304b of FIG. 1), or to receive multiple elongated devices 300 (e.g., both of the lead bodies 106 of FIG. 1), or both.

A lead extension connector 322 is disposed on the lead extension 324. In FIG. 2B, the lead extension connector 322 is shown disposed at a distal portion 326 of the lead extension 324. The lead extension connector 322 includes a connector housing 328. The connector housing 328 defines at least one port 330 into which terminals 310 of the elongated device 300 can be inserted, as shown by directional arrow 338. Each of the terminals 310 can couple to the light emitter 135 or one or more of the electrodes 134. The connector housing 328 also includes a plurality of connector contacts, such as connector contact 340. When the elongated device 300 is inserted into the port 330, the connector contacts 340 disposed in the connector housing 328 can be aligned with the terminals 310 of the elongated device 300 to electrically couple the lead extension 324 to the electrodes (134 of FIG. 1) disposed along the lead (103 in FIG. 1).

In at least some embodiments, the proximal end of the lead extension 324 is similarly configured and arranged as a proximal end of the lead 103 (or other elongated device 300). The lead extension 324 may include a plurality of electrically-conductive wires (not shown) that electrically couple the connector contacts 340 to a proximal portion 348 of the lead extension 324 that is opposite to the distal portion 326. In at least some embodiments, the conductive wires disposed in the lead extension 324 can be electrically coupled to a plurality of terminals (not shown) disposed along the proximal portion 348 of the lead extension 324. In at least some embodiments, the proximal portion 348 of the lead extension 324 is configured and arranged for insertion into a connector disposed in another lead extension (or another intermediate device). In other embodiments (and as shown in FIG. 2B), the proximal portion 348 of the lead extension 324 is configured and arranged for insertion into the control module connector 144.

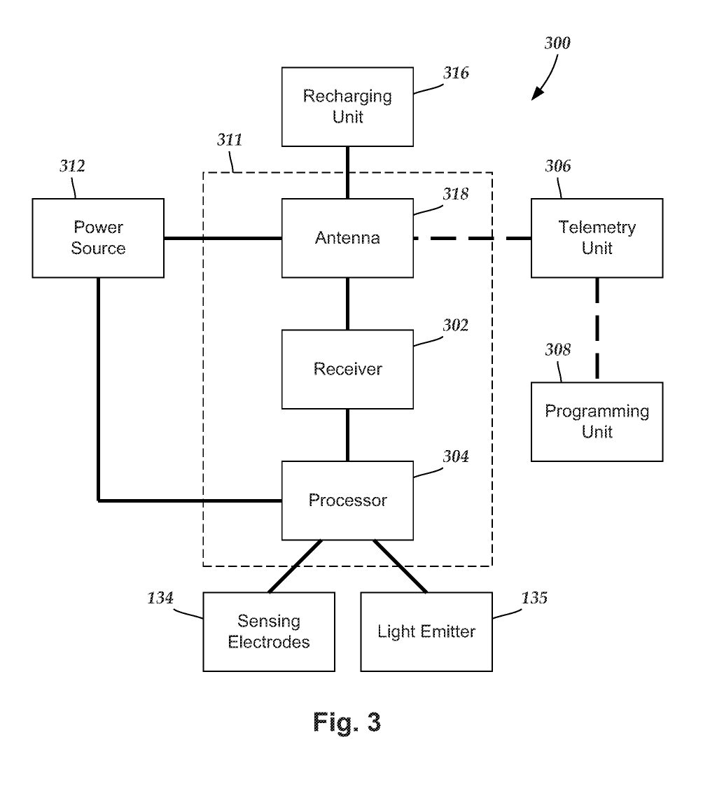

FIG. 3 is a schematic overview of one embodiment of components of an optical stimulation system 300 including an electronic subassembly 311 disposed within a control module. It will be understood that the optical stimulation system can include more, fewer, or different components and can have a variety of different configurations including those configurations disclosed in the stimulator references cited herein.

Some of the components (for example, a power source 312, an antenna 318, a receiver 302, and a processor 304) of the optical stimulation system can be positioned on one or more circuit boards or similar carriers within a sealed housing of an implantable pulse generator, if desired. Any power source 312 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 318 or a secondary antenna. The external power source can be in a device that is mounted on the skin of the user or in a unit that is provided near the user on a permanent or periodic basis.

If the power source 312 is a rechargeable battery, the battery may be recharged using the optional antenna 318, if desired. Power can be provided to the battery for recharging by inductively coupling the battery through the antenna to a recharging unit 316 external to the user. Examples of such arrangements can be found in the references identified above.

In one embodiment, light is emitted by the light emitter 135 of the lead body to stimulate nerve fibers, muscle fibers, or other body tissues near the optical stimulation system. The processor 304 is generally included to control the timing and other characteristics of the optical stimulation system. For example, the processor 304 can, if desired, control one or more of the intensity, wavelength, amplitude, pulse width, pulse frequency, cycling (e.g., for repeating intervals of time, determining how long to stimulate and how long to not stimulate), and electrode stimulation configuration (e.g., determining electrode polarity and fractionalization) of the optical stimulation.

Additionally, the processor 304 can select which, if not all, of the sensing electrodes are activated. Moreover, the processor 394 can control which types of signals the sensing electrodes detect. In at least some embodiments, the sensing electrodes detect a level of neuronal activation, or neuronal firing rates, or both, received directly from the target stimulation location. In other embodiments, the sensing electrodes detect one or more other signals received from the target stimulation location in addition to, or in lieu of the level of neuronal activation or neuronal firing rates, such as evoked compound action potentials, local field potentials, multiunit activity, electroencephalograms, electrophysiology, or electroneurograms. In at least some embodiments, one or more of the received signals (e.g., evoked compound action potentials, local field potentials, multiunit activity, electroencephalograms, electrophysiology, electroneurograms, or the like) can be used to indirectly measure the level of neuronal activation, or neuronal firing rates, or both, at the target stimulation location.

Optionally, the processor 304 can select one or more stimulation electrodes to provide electrical stimulation, if desired. In some embodiments, the processor 304 selects which of the optional stimulation electrode(s) are cathodes and which electrode(s) are anodes.

Any processor can be used and can be as simple as an electronic device that, for example, produces optical stimulation at a regular interval or the processor can be capable of receiving and interpreting instructions from an external programming unit 308 that, for example, allows modification of stimulation characteristics. In the illustrated embodiment, the processor 304 is coupled to a receiver 302 which, in turn, is coupled to the optional antenna 318. This allows the processor 304 to receive instructions from an external source to, for example, direct the stimulation characteristics and the selection of electrodes, if desired.

In one embodiment, the antenna 318 is capable of receiving signals (e.g., RF signals) from an external telemetry unit 306 which is programmed by the programming unit 308. The programming unit 308 can be external to, or part of, the telemetry unit 306. The telemetry unit 306 can be a device that is worn on the skin of the user or can be carried by the user and can have a form similar to a pager, cellular phone, or remote control, if desired. As another alternative, the telemetry unit 306 may not be worn or carried by the user but may only be available at a home station or at a clinician's office. The programming unit 308 can be any unit that can provide information to the telemetry unit 306 for transmission to the optical stimulation system 300. The programming unit 308 can be part of the telemetry unit 306 or can provide signals or information to the telemetry unit 306 via a wireless or wired connection. One example of a suitable programming unit is a computer operated by the user or clinician to send signals to the telemetry unit 306.

The signals sent to the processor 304 via the antenna 318 and the receiver 302 can be used to modify or otherwise direct the operation of the optical stimulation system. For example, the signals may be used to modify the stimulation characteristics of the optical stimulation system such as modifying one or more of stimulation duration, pulse frequency, waveform, and stimulation amplitude. The signals may also direct the optical stimulation system 300 to cease operation, to start operation, to start charging the battery, or to stop charging the battery. In other embodiments, the stimulation system does not include the antenna 318 or receiver 302 and the processor 304 operates as programmed.

Optionally, the optical stimulation system 300 may include a transmitter (not shown) coupled to the processor 304 and the antenna 318 for transmitting signals back to the telemetry unit 306 or another unit capable of receiving the signals. For example, the optical stimulation system 300 may transmit signals indicating whether the optical stimulation system 300 is operating properly or not or indicating when the battery needs to be charged or the level of charge remaining in the battery. The processor 304 may also be capable of transmitting information about the stimulation characteristics so that a user or clinician can determine or verify the characteristics.

Turning to FIG. 4, optogenetics is a type of optical stimulation that uses light to control, measure, or monitor activities of neurons into which one or more genetic agents have been introduced. The introduced genetic agents cause a measurable effect in the neurons (e.g., excitation, inhibition) when optically stimulated at certain wavelengths. Cells that have not received the genetic agent typically do not elicit a similar effect from the optical stimulation as cells that receive the genetic agents. In some instances, cells that have not received the genetic agent may elicit a smaller (e.g., subthreshold) effect from the optical stimulation than cells that receive the genetic agents.

Any suitable technique can be used for introducing the genetic agent(s) to cells at a target stimulation location including, for example, transduction, transfection, or both. In at least some embodiments, the genetic agents are introduced into cells using viral vectors. Delivery of the genetic agent(s) can be intravenously, intracranially, or the like or combinations thereof. Optogenetics can be used to provide therapy for a variety of different disorders or conditions including, for example, chronic pain, spinal cord injury sensory function (e.g., transfecting sensory neurons to reactivate them), spinal cord injury motor function (e.g., transfecting sensory neurons to reactivate them), chronic itch, inflammatory pain (e.g., arthritis), pain associated with cancer, overactive bladder, incontinence, sexual dysfunction following spinal cord injury/neuropathy, diabetic neuropathy/peripheral neuropathy, multiple sclerosis, and other disorders or conditions that might have a peripheral/spinal etiology which could be modulated by controlling the activity of spinal sensory or motor neurons.

Optogenetics may provide advantages over electrical stimulation. Optogenetics may provide increased specificity of stimulation, as compared to electrical stimulation. For example, a light emitter may be much smaller in size than an implanted electrical stimulation electrode. Optical stimulation specificity may be further affected by other factors, such as absorbance of light, the amount/uptake of introduced genetic agents, inhibition in and around the target optical stimulation location. Accordingly, the region of tissue stimulated by optical stimulation may be much smaller in size than a region of tissue stimulated by electrical stimulation. Increased specificity of stimulation at a target location may potentially reduce undesired side effects caused by collateral stimulation of untargeted patient tissue.

Additionally, optogenetics can enable concurrent sensing/recording of electrical activity (e.g., neural activity, such as a level of neuronal activation or neuronal firing rates) during stimulation. In contrast, electrical stimulation may mask base-line electrical activity because the current needed to depolarize cells at a target stimulation location may obscure the base-line electrical activity within (or in proximity to) the target stimulation location.

Light-sensitive neurons have at least one channel, tertiary protein structure, etc. that undergoes a distinct conformal, physiological, electrophysiological, and/or electrical change of at least a portion of the neuron in response to one or more specific wavelengths of light. Genetic agent(s) introduced into the cells can encode for one or more light-sensitive proteins, such as opsins, related to the production of ion channels. The encoded light-sensitive proteins are activated (e.g. stimulated to open or close a channel, drive a pump to raise or lower the membrane potential of a cell, or the like) within a particular range of wavelengths.

Suitable light-sensitive proteins include, for example, channelrhodopsins, halorhodopsins, archaerhodopsins, or other ion-channel-related proteins. The particular wavelength ranges over which the encoded proteins are activated may be different for different proteins. In at least some embodiments, channelrhodopsin is responsive in the range of 425 nm-475 nm, while halorhodopsin is responsive in range of 550 nm-600 nm. The activation wavelength ranges for different genetic agents may, or may not, overlap with one another.

Suitable target stimulation locations include, but are not limited to, at least one of the patient's brain, spinal cord, cauda equina, one or more dorsal root entry zones, one or more dendritic cells, one or more dorsal root ganglia, or one or more spinothalamic tracts, peripheral sensory and motor nerves, peripheral plexi (e.g. brachial, solar, mesenteric, and the like), peripheral receptors, free nerve endings, rootlets, distal axons of dorsal root ganglia (peripheral nerves), dorsal columns.

In at least some embodiments, genetic agents are delivered to multiple target stimulation locations (e.g., dorsal root ganglion and dendritic cells) from the same location either concurrently or sequentially.

The optical stimulation lead can be positioned in proximity to the target stimulation location(s) before, during, or after introduction of the genetic agent(s) into cells of the target stimulation location. In some embodiments, one or more excitatory genetic agents are exclusively delivered to cells. In other embodiments, one or more inhibitory genetic agents are exclusively delivered to cells. In at least some embodiments, multiple types of genetic agents are delivered to cells. In some instances, the delivered genetic agents include at least one type of excitatory genetic agent and at least one type of inhibitory genetic agent, where the excitatory genetic agent and the inhibitory genetic agent are activated at different wavelengths, or ranges of wavelengths. In at least some embodiments, an excitatory agent and an inhibitory agent are delivered into cells together. For example, an excitatory agent and an inhibitory agent can be part of the same viral vector.

At some point after expression of the genetic agents begins within the cells at the target stimulation location, light is emitted by the optical stimulation lead towards the target stimulation location from a position in proximity to the target stimulation location. Light is emitted via the one or more light emitters. In at least some embodiments, one or more electrical signals output from neurons within the target stimulation location are sensed by one or more sensing electrodes.

FIG. 4 schematically shows one embodiment of an optical lead system 400 that includes a lead 403 with a lead body 406. Optical fibers 420a, 420b disposed in the lead 403 couple light emitters 435a, 435b, respectively, disposed along a distal portion 426 of the lead 403 to a light source 411 (for generating light) and a processor 404 (for applying one or more stimulation parameters to the generated light, turning off one or more of the light emitters, or the like). The light emitters 435a, 435b may, optionally, be disposed beneath optically-transparent regions 470a, 470b, respectively, through which light emitted from the light emitters passes. Sensing electrodes 434 are disposed along the distal portion 426 of the lead and are also coupled to the processor 404 via one or more electrical conductors (not shown).

The light source 411 generates the light emitted by the light emitters 435a, 435b. Optionally, the light is passed through one or more optical components 414 (e.g., collimators, optical lenses, optical filters, or the like) to alter characteristics of the light prior to emission from the light emitters 435a, 435b. In the illustrated embodiment, the optical components 414 are shown positioned between the light source 411 and the processor 404. It will be understood that the one or more optical components 414 can, alternatively or additionally, be disposed between the processor 404 and the lead 403, along the exterior of the lead body 406, embedded within the lead body, or any combination thereof.

The light generated by the light source can be within any suitable range of wavelengths for providing optical therapy, including infrared, visible, or ultraviolet wavelengths. In at least some embodiments, the light is emitted in one or more narrow bands of wavelengths (e.g., a band having a range of no more than 100 nm, 50 nm, 25 nm, 20 nm, 15 nm, 10 nm, or 5 nm). In at least some embodiments, the wavelengths are no less than 400 nm. In at least some embodiments, the wavelengths are no greater than 650 nm. In at least some embodiments, the wavelengths are no less than 425 nm and no greater than 600 nm. In at least some embodiment, the wavelengths are no less than 425 nm and no greater than 475 nm. In at least some embodiment, the wavelengths are no less than 550 nm and no greater than 600 nm.

The illustrated embodiment shows two optical fibers 420a, 420b. Any suitable number of optical fibers may be utilized including, one, two, three, four, five, six, seven, eight, nine, ten, eleven, twelve, fourteen, sixteen, eighteen, twenty, thirty, or more optical fibers. In at least some embodiments, there is an optical fiber for every light emitter.

As an alternative to the optical fibers, one or more light emitting diodes (LEDs), organic light emitting diodes (OLEDs), laser diodes, or other light sources may be disposed along the distal portion of the lead to provide the light. For example, one or more white light sources can be disposed along the distal portion of the lead. Alternatively, one or more light sources for each of multiple colors, wavelengths, or wavelength bands can be disposed along the distal portion of the lead. These light sources can be electrically coupled to the control module by conductors that extend along the lead. The control module can then direct turning on and off the light sources, leads (if multiple leads are implanted), as well as other stimulation parameters such as intensity, wavelength, amplitude, pulse width, pulse frequency, cycling, electrode stimulation configuration, and the like using signals sent to the light source(s) over the conductors.

Light is emitted to the target stimulation location(s) via the one or more light emitters. In the illustrated embodiment, the light emitter 435a is disposed along a side of the lead and is side-facing (i.e., light is emitted outwardly from a side of the lead), and the light emitter 435b is disposed at a distal tip of the lead and is forward-facing (i.e., light is emitted distally outwardly from the distal tip of the lead). Alternatively, all of the light emitters can be side-facing, or all of the light emitters can be forward-facing. In at least some embodiments, therapy is directed towards two or more target stimulation locations that are stimulated concurrently or sequentially from the same lead position. In which case, one or more of the light emitters can be directed to one of the target stimulation locations, while one or more of the remaining light emitters are directed to a different target stimulation location. In at least some embodiments, multiple light emitters are directed towards the same target stimulation location.

As mentioned above, some genetic agents delivered to cells cause an excitatory response, while others cause an inhibitory response. The wavelengths at which the particular genetic agents are activated may be different. Thus, a first stimulation wavelength may activate a first genetic agent that generates an excitatory response, while a second stimulation wavelength that is different than the first stimulation wavelength may activate a second genetic agent that generates an inhibitory response.

Accordingly, activation by optical stimulation can cause neurons to become excited or become inhibited, depending on which type of genetic agent is introduced into those neurons, and which wavelengths of light are used to stimulate those neurons. In some instances, both excitatory and inhibitory genetic agents are introduced into the same neurons. In which case, selectively switching between an excitatory range of wavelengths and an inhibitory range of wavelengths (i.e., steering) can be used to elicit either an excitatory response or an inhibitory response from those neurons.

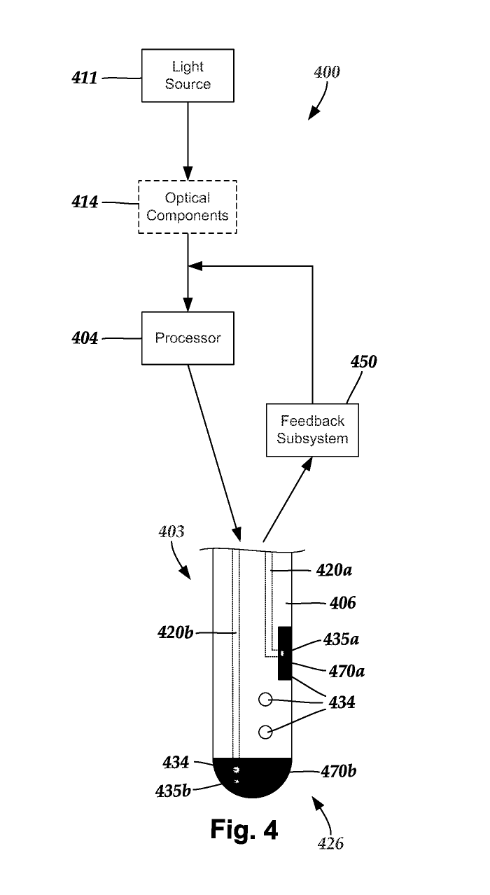

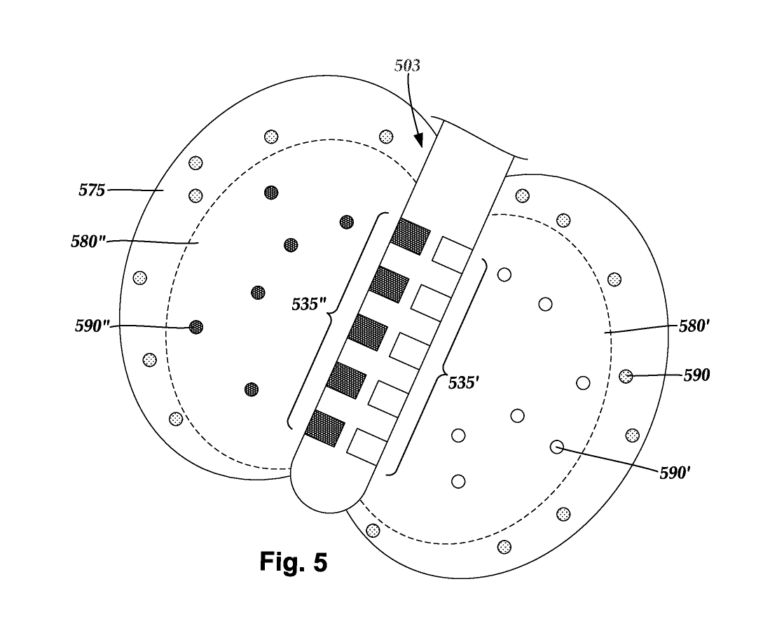

FIG. 5 shows one embodiment of a distal portion of a lead 503 disposed within a target stimulation location 575. Multiple neurons (indicated as lightly-stippled circles), such as neuron 590, are disposed in the target stimulation location 575. Both excitatory and inhibitory genetic agents have been introduced into the neurons within the target stimulation location 575, such that neurons can either be inhibited by light emitted at a first activation wavelength or excited by light emitted at a second activation wavelength.

Light emitters are disposed along opposing sides of the lead. In FIG. 5, and in other figures, light emitters configured for emitting light at an inhibitory activation wavelength, such as light emitters 535′ in FIG. 5, are shown in solid white and are hereinafter referred to as “inhibiting emitters”, while light emitters configured for emitting light at an excitatory activation wavelength, such as light emitters 535″ in FIG. 5, are shown as heavily stippled and are hereinafter referred to as “exciting emitters”. In some embodiments, the light emitters can be individually programmed to emit light at either the first wavelength or the second wavelength. In some embodiments, the light emitters can also be individually programmed to turn off. Individually adjusting the light emitters to be inhibiting, exciting, or off, can potentially change the sizes, shapes, and locations of the activation volumes.

In the illustrated embodiment, a first activation volume 580′ is shown extending generally outwards from the inhibiting emitters 535′ in response to light emitted at the first activation wavelength. Neurons 590′ within the first activation volume 580′ are inhibited, as indicated by no stippling, while neurons that are inside the target stimulation location 575 yet outside of the first activation volume 508′, are not inhibited. A second activation volume 580″ is shown extending generally outwards from the exciting emitters 535″ in response to light emitted at the second activation wavelength. Neurons 590″ within the second activation volume 580″ are excited, as indicated by heavy stippling, while neurons that are inside the target stimulation location 575 yet outside of the second activation volume 580″, are not excited.

Turning back to FIG. 4, the sizes and shapes of the activation volumes are influenced by the stimulation parameters of the emitted light. The sizes and shapes obtained using a given set of stimulation parameters are sensed using sensing electrodes. The one or more sensing electrodes 434 are disposed along the distal portion of the lead and adapted to sense one or more electric signals. The electric signals can include background signals, signals emitted in response to optical stimulation, or both. The one or more sensed electrical signals can include sensing changes in electrical activity in at least some cells within the target stimulation location in response to the optical stimulation. The sensing electrodes can be adapted to sense various different types of signals from targeted cell populations including, for example, one or more of sensing a level of neuronal activation, or neuronal firing rates, or both.

Signals from targeted cell populations can be sensed directly, or indirectly (i.e., a surrogate) using any electrical signal recordable from the nervous system that indicates neural activity. Suitable surrogate signals include, for example, evoked compound action potentials, local field potentials, multiunit activity signals (e.g., determining neuronal firing rates by counting spikes per unit of time), electroneurogram signals (e.g., measuring activity in peripheral nerves based on a response-to-noise ratio), electroencephalogram signals, electrophysiology signals, or the like or combinations thereof received from the target stimulation location. In at least some embodiments, the changes in the sensed signals correspond to one or more disorders or conditions of interest.

As shown in FIG. 4, a closed-loop feedback subsystem 450 couples the processor 404 to the sensing electrodes 434. Electrical signals sensed from the sensing electrodes may provide information about the sizes and shapes of the activation volumes which, in turn, can be used to adjustment stimulation to improve therapy. Accordingly, the closed-loop feedback subsystem 450 can be used to adjust one or more parameters of the emitted light (e.g., intensity, wavelength, amplitude, pulse width, pulse frequency, cycling, electrode stimulation configuration, and the like) based on the sensed electrical signals (e.g., sensing of a new signal, sensing a change in the amount or quality of a signal, the disappearance of a signal, or the like).

The closed-loop feedback subsystem can be implemented in a variety of different ways including, for example, as a proportional controller, a proportional integral controller, a proportional derivative controller, or a proportional-integral-derivative controller. In at least some embodiments, the closed-loop feedback controller is a hybrid controller that includes smart machine learning module. The closed-loop feedback subsystem 450 can be implemented by the processor 404, or can be a stand-alone controller.

The controller could be an adaptive (in a deterministic way)/Kalman filter whose gain and transfer function can change according to user setting, symptom severity, and the nature/amplitude of the signals being measured, among other parameters. In at least some embodiments, the controller is used to adjust optical signals, electrical signals, or both, being delivered to the patient via the stimulation system.

The one or more sensing electrodes can be disposed at any location suitable for sensing and recording electrical activity from cells at the target stimulation location. The sensing electrodes can be disposed along the lead body. In some embodiments, the sensing electrodes are disposed along one or more optically-transparent regions of the lead body. In some instances, one or more of the sensing electrodes are disposed on, or in proximity to, one or more of the light emitters. In some instances, one or more of the sensing electrodes are disposed on, or in proximity to, one or more of the optical fibers.

In the embodiment illustrated in FIG. 4, sensing electrodes 434 are shown disposed in the lead body 406, and also on the optical fiber 420b in proximity to the forward-facing light emitter 435b (and aligned with the distal-tip optically-transparent region 470b). Additionally, the embodiment illustrated in FIG. 4 shows one of the sensing electrodes formed as a transparent material disposed along the segmented optically-transparent region 470a. It will be understood that, in various embodiments, an optical stimulation lead assembly can include one or more sensing electrodes disposed at any suitable location along one or more optical fibers, the lead body, one or more optically-transparent regions, or any combination thereof.

In some embodiments, the number of sensing electrodes of a lead assembly is equal to the number of light emitters. In some embodiments, the number of sensing electrodes of a lead assembly is greater than the number of light emitters. In other embodiments, the number of sensing electrodes of a lead assembly is fewer than the number of light emitters.

The light emitter(s) and sensing electrode(s) can be disposed on any implantable lead suitable for emitting light and sensing electrical activity. In the embodiment illustrated in FIG. 4, the light emitter(s) and sensing electrode(s) are shown disposed along a percutaneous lead. It will be understood that the light emitter(s) and sensing electrode(s) can be disposed along other types of lead including, for example, paddle lead, cuff leads, or the like. FIG. 4 shows a single lead. It will be understood that an optical stimulation system can include multiple leads, with at least one light emitter disposed along each of the leads. In some instances, a sensing electrode disposed along a first lead may sense electrical activity in response to stimulation from a second lead.

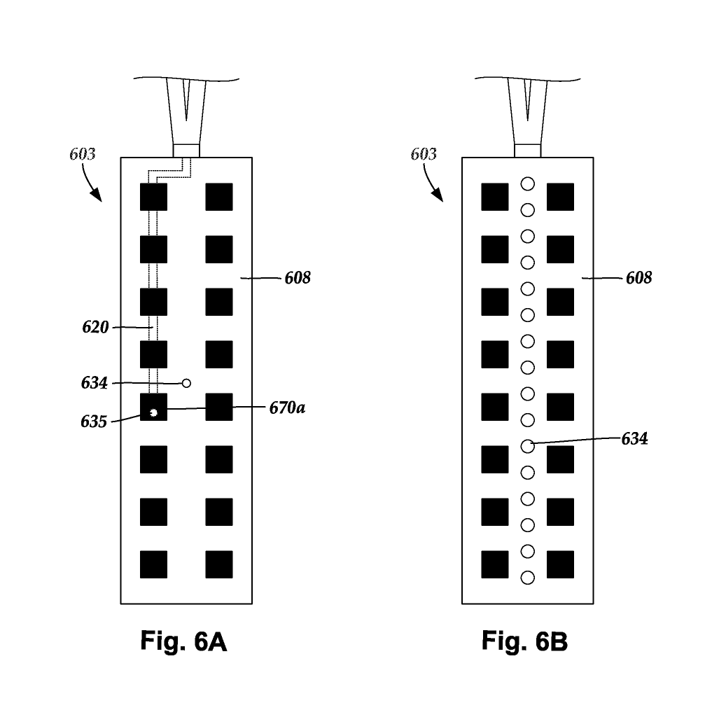

FIGS. 6A-6B illustrate several embodiments of light emitters and sensing electrodes disposed along a paddle lead. FIG. 6A shows a distal portion of a paddle lead 603 suitable for implantation. The paddle lead 603 includes multiple light emitters, such as light emitter 635, and a sensing electrode 634 disposed along a paddle body 608. In the embodiment illustrated in FIG. 6A, an optional optically-transparent region 670a is shown formed into the paddle body 608 over the light emitter 635. In at least some embodiments, an optically-transparent region is disposed over each of the light emitters. In alternate embodiments, the lead does not include optically-transparent regions.

In the embodiment illustrated in FIG. 6A, the light emitter 635 is shown disposed along an optical fiber 620. In at least some embodiments, each light emitter is disposed along a different optical fiber (not shown). In alternate embodiments, light emitted from light emitters is generated from a light source disposed in the paddle body and there are no optical fibers coupling the light emitters to a remote light source. In FIG. 6A, the light emitters are shown arranged in a 2×8 configuration. Many other configurations are possible including, for example, 1×8, 4×8, 2×4, 4×4, or other configurations.

The sensing electrodes can be disposed along any suitable portion of the paddle body 608. In FIG. 6A, the sensing electrode is shown schematically as a circle disposed along a central portion of the paddle body 608. In at least some embodiments, one or more sensing electrodes are disposed along at least one of the optically-transparent regions. In at least some embodiments, one or more sensing electrodes are disposed along an optical fiber.

A lead can include any suitable number of sensing electrodes for sensing electrical activity. FIG. 6B shows an alternate embodiment of the paddle lead 603 where multiple sensing electrodes, such as sensing electrode 634, are arranged longitudinally along the paddle body 608 in a single column between columns of the light emitters.

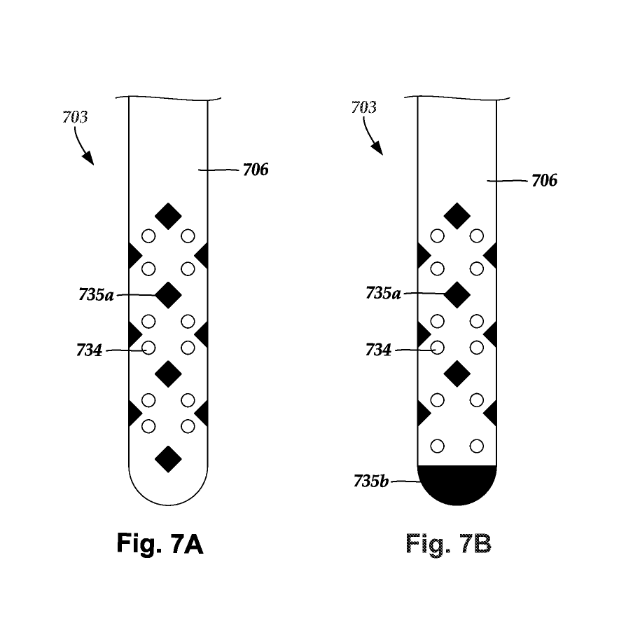

Many different light emitter and sensing electrode configurations are possible. FIGS. 7A-9B illustrate a few exemplary arrangements of light emitters and sensing electrode configurations disposed along percutaneous leads. In the leads of 7A-9B, the sensing electrodes are shown schematically as circles. Optionally, the sensing electrodes can be formed as segmented electrodes, ring-shaped electrodes, distal tip electrodes, or any other shape suitable for disposing along a lead and sensing electrical activity. The sensing electrodes can be side-facing or forward-facing. In some embodiments, the sensing electrodes have larger surface areas than the light emitters. In other embodiments, the sensing electrodes have smaller surface areas than the light emitters.

FIG. 7A shows a distal portion of a lead 703 having a lead body 706 with light emitters, such as light emitter 735a, disposed along a segmented cutout in the lead body 706, and sensing electrodes, such as sensing electrode 734, disposed along the lead body 706. In FIG. 7A, the light emitters are all side-facing and arranged into configurations that each extends along less than 50% of a circumference of the lead 703, such that each light emitter emits light from the lead along a narrow arc and does not direct light evenly around a circumference of the lead at a particular axial position along a longitudinal length of the lead. Such an arrangement enables light to be targeted exclusively to a particular region around the circumference of the lead. In the illustrated embodiment, the segmented cutouts are diamond-shaped. Other shapes are possible including, for example, square, rectangular, triangular, pentagonal, round, oval, capsule-shaped, or other geometric or non-geometric shapes.

FIG. 7B shows a distal portion of the lead 703 with segmented light emitters, such as segmented light emitter 735a, and sensing electrodes, such as sensing electrode 734, disposed along the lead body 706. The embodiment illustrated in FIG. 7B additionally includes a distal-tip light emitter 735b disposed at a distal tip of the lead 703. The distal-tip light emitter 735b enables the light emitter to emit light distally from the distal tip of the lead 703 (i.e., a forward-facing light emitter).

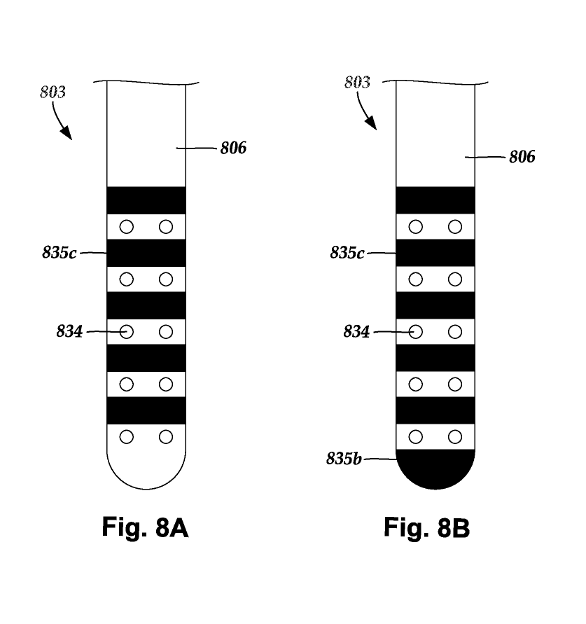

FIG. 8A shows a distal portion of a lead 803 having a lead body 806 with ring-shaped light emitters, such as ring-shaped light emitter 835c, and sensing electrodes, such as sensing electrode 834, disposed along the lead body 806. In FIG. 8A, the light emitters are all side-facing and arranged into rings extending around an entire, or substantially-entire, circumference of the lead body 806, such that the light emitter can direct light evenly, or substantially-evenly, around 50%, 60%, 70%, 80%, 90%, or 100% of the circumference of the lead body 806 at a particular axial position along a longitudinal length of the lead.

The embodiment illustrated in FIG. 8B additionally includes a distal-tip light emitter 835b disposed at a distal tip of the lead 803. The distal-tip light emitter 835b enables the light emitter to emit light distally from the distal tip of the lead 803 (i.e., a forward-facing light emitter). It will be understood that, in various embodiments, the lead 803 includes one or more sensing electrodes disposed along one or more optical fibers, one or more optically-transparent regions, or any combination thereof, in lieu of, or in addition to, being disposed along the lead body 806.

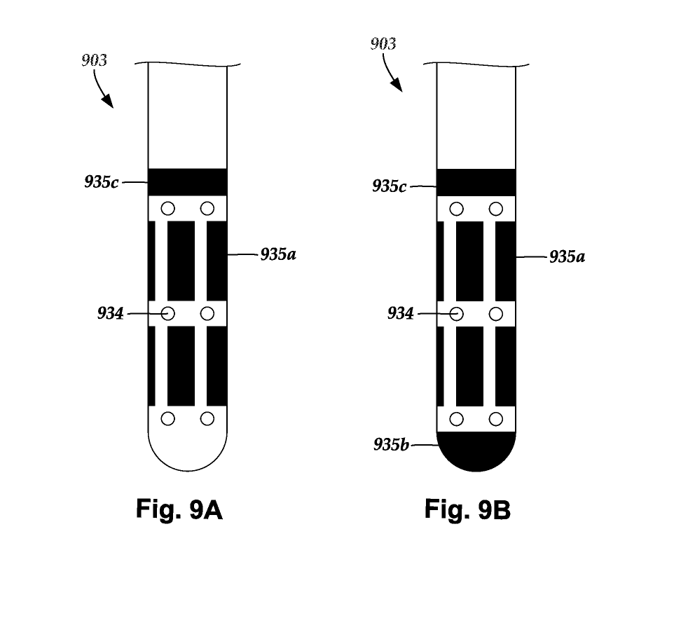

FIG. 9A shows a distal portion of a lead 903 having a lead body 906 with light emitters, such as segmented light emitter 935a and ring-shaped light emitter 935c, and sensing electrodes, such as sensing electrode 934, disposed along the lead body 906. FIG. 9B additionally includes a distal-tip light emitter 935b disposed at a distal tip of the lead 903. The light emitter 935b enables light to be emitted distally from the distal tip of the lead 903.

The methods and systems described herein may be embodied in many different forms and should not be construed as limited to the embodiments set forth herein. Accordingly, the methods and systems described herein may take the form of an entirely hardware embodiment, an entirely software embodiment or an embodiment combining software and hardware aspects. Systems referenced herein typically include memory and typically include methods for communication with other devices including mobile devices. Methods of communication can include both wired and wireless (e.g., RF, optical, or infrared) communications methods and such methods provide another type of computer readable media; namely communication media. Wired communication can include communication over a twisted pair, coaxial cable, fiber optics, wave guides, or the like, or any combination thereof. Wireless communication can include RF, infrared, acoustic, near field communication, Bluetooth™, or the like, or any combination thereof.

It will be understood that each of the methods disclosed herein, can be implemented by computer program instructions. These program instructions may be provided to a processor to produce a machine, such that the instructions, which execute on the processor, create means for implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An optical stimulation system, comprising:
an optical stimulation lead comprising
a lead body having a distal portion and a proximal portion,
a light emitter disposed along the distal portion of the lead body and configured and arranged to emit light having one or more wavelengths that activate light-sensitive neurons within a target stimulation location, the light-sensitive neurons generating either an excitatory response or an inhibitory response when activated depending on the wavelength of the emitted light, and
a sensing electrode disposed along the distal portion of the lead body, the sensing electrode configured and arranged to sense electrical activity from the activated light-sensitive neurons concurrently with emission of the light from the light emitter; and
a control module coupleable to the optical stimulation lead, the control module configured and arranged to direct the emission of the light from the light emitter using a set of stimulation parameters, the control module comprising a closed-loop feedback subsystem configured and arranged for adjusting at least one stimulation parameter of the set of stimulation parameters based, at least in part, on electrical activity of the activated light-sensitive neurons sensed by the sensing electrode.

2. The optical stimulation system of claim 1, wherein the sensing electrode is configured and arranged to sense changes in electrical activity from the activated light-sensitive neurons in response to the emitted light.

3. The optical stimulation system of claim 1, wherein the sensing electrode is configured and arranged to sense at least one of a level of neuronal activation or a neuronal firing rate of the light-sensitive neurons in response to the emitted light.

4. The optical stimulation system of claim 3, wherein the sensing electrode is configured and arranged to sense at least one surrogate electrical signal from the light-sensitive neurons in response to the emitted light, the surrogate electrical signal usable for determining at least one of a level of neuronal activation or a neuronal firing rate of the light-sensitive neurons in response to the emitted light.

5. The optical stimulation system of claim 4, wherein the at least one surrogate electrical signal comprises one of an evoked a compound action potential, local field potential, a multiunit activity signal, an electroencephalogram signal, an electrophysiology signal, an electrospinogram signal, or an electroneurogram signal.

6. The optical stimulation system of claim 1, wherein the set of stimulation parameters comprises at least one of intensity, pulse width, pulse frequency, cycling, electrode stimulation configuration.

7. The optical stimulation system of claim 1, wherein the closed-loop feedback subsystem comprises one of a proportional controller, a proportional integral controller, a proportional derivative controller, or a proportional-integral-derivative controller.

8. The optical stimulation system of claim 1, wherein the closed-loop feedback subsystem comprises a smart machine learning module.

9. The optical stimulation system of claim 1, wherein the light emitter is side-facing with respect to the lead body.

10. The optical stimulation system of claim 1, wherein the light emitter is forward-facing with respect to the lead body.

11. The optical stimulation system of claim 1, further comprising a light source in communication with the control module, the light source configured and arranged to generate light emitted by the light emitter.

12. The optical stimulation system of claim 1, wherein the light source is disposed in the control module.

13. The optical stimulation system of claim 1, wherein the light source is disposed in the lead.

14. The optical stimulation system of claim 1, further comprising a programmer coupled to the control module, the programmer configured and arranged for implementing at least one stimulation parameter of the set of stimulation parameters.

15. A method for optically stimulating a patient, the method comprising:
advancing the optical stimulation lead of the optical stimulation system of claim 1 in proximity to a first target stimulation location within the patient, the first target stimulation location containing light-sensitive neurons, the light-sensitive neurons generating either an excitatory response when activated by light of a first wavelength, or an inhibitory response when activated by light of a second wavelength;
emitting light at either the first wavelength or the second wavelength from the light emitter of the optical stimulation lead towards the first target stimulation location;
sensing, using the sensing electrode of the optical stimulation lead, electrical activity from the light-sensitive neurons at the first target stimulation location; and
adjusting, using the closed-loop feedback subsystem of the optical stimulation system, at least one stimulation parameter of the set of stimulation parameters of the emitted light in response to changes in electrical activity sensed by the sensing electrode.

16. The method of claim 15, wherein adjusting, using the closed-loop feedback subsystem of the optical stimulation system, at least one stimulation parameter of the set of stimulation parameters of the emitted light in response to changes in electrical activity sensed by the sensing electrode comprises adjusting intensity of the emitted light.

17. The method of claim 15, wherein sensing, using the sensing electrode of the optical stimulation lead, electrical activity from the light-sensitive neurons at the first target stimulation location comprises sensing at least one of a level of neuronal activation or neuronal firing rate of the light-sensitive neurons within the first target stimulation location.

18. The method of claim 15, wherein sensing, using the sensing electrode of the optical stimulation lead, electrical activity from the light-sensitive neurons at the first target stimulation location comprises sensing at least one of an evoked compound action potential, a local field potential, a multiunit activity signal, an electroencephalogram signal, an electrophysiology signal, an electrospinogram signal, or an electroneurogram signal within the first target stimulation location.

19. The method of claim 15, wherein sensing, using the sensing electrode of the optical stimulation lead, electrical activity from the light-sensitive neurons at the first target stimulation location comprises sensing electrical activity from the light-sensitive neurons at the first target stimulation location concurrently with emission of light from the light emitter.

20. The method of claim 15, wherein advancing the optical stimulation lead of the optical stimulation system of claim 1 in proximity to a first target stimulation location within the patient comprises
advancing the optical stimulation lead in proximity to each of a first target stimulation location and a second target stimulation location within the patient, the first target stimulation location and the second target stimulation location each containing light-sensitive neurons, the light-sensitive neurons generating either an excitatory response when activated by light of a first wavelength, or an inhibitory response when activated by light of a second wavelength, and
emitting, concurrently, light at either the first wavelength or the second wavelength from the light emitter of the optical stimulation lead towards the first target stimulation location and the second target stimulation location.
